(19) United States
(12) Patent Application Publication
(10) Pub. No.: US 2014/0330272 A1
(43) Pub. Date: Nov. 6, 2014

(54) APPLICATION PROBE

(71) Applicant: OLYMPUS WINTER & IBE GMBH, Hamburg (DE)

(72) Inventor: German KLINK, Berlin (DE)

(21) Appl. No.: 14/269,702

(22) Filed: May 5, 2014

(30) Foreign Application Priority Data
May 3, 2013 (DE) 10 2013 208 167.7

(51) Int. Cl. A61B 18/14

(52) U.S. Cl. CPC A61B 18/1492; A61B 2018/00541
USPC 606/46

(57) ABSTRACT

The invention relates to an application probe in which the probe is used to apply radiofrequency alternating current to surrounding tissue, wherein the application probe includes a shank with a distal shank part, which has shank longitudinal sections movable relative to one another. The distal shank part formed thus can be pliable in a first state, as a result of which the application probe can be guided to the application location thereof through an endoscope. In a second state, the shank longitudinal sections of the distal shank part are braced to one another by a pulling element contained in the application probe and form a rigid probe end, which can be employed, together with a suitable probe tip, to penetrate. Moreover, the shank longitudinal sections form the electrode(s) of the application probe for applying the alternating current to surrounding tissue.

The invention relates to an application probe, in particular for an interstitial application, in which the probe is used to apply radiofrequency alternating current to surrounding tissue.

In principle, such application probes are known and can e.g. have a needle-shaped embodiment such that they penetrate tissue transcutaneously. By way of example, one or two electrodes can be situated in the region of the distal end of such an application probe, by means of which electrodes radiofrequency electric current can be applied to surrounding tissue. Monopolar application probes require only one electrode. During the application, this one electrode interacts with a large-area return or neutral electrode, which is likewise in contact with the body of a patient.

For a bipolar application, provision is made for application probes with at least two electrodes, wherein, during use, the current flows e.g. between the two probe electrodes of an application probe, but optionally also between probe electrodes of different application probes.

Needle-shaped application probes embodied for penetration into tissue are typically sufficiently rigid and often have a pointed tip, for example with trocar grinding, since this makes penetration into tissue easier.

In addition to such relatively rigid application probes, application probes which are comparatively easy to bend and can be bent similarly to a tube, a cable or string, are also known. By way of example, such application probes are used within vein ablation for the treatment of varicose veins.

A further field of use of application probes lies in those applications, in which a bendable or pliable application probe is led through the work channel of e.g. an endoscope and finally emerges at the distal end of this work channel. By way of example, such an application probe is pushed through a work channel within the scope of the treatment of bronchial carcinomas, it emerges at the end of said work channel and it is then intended to penetrate e.g. a bronchial wall, which may be cartilaginous. For such applications, application probes, which are bendable or pliable per se, can also be provided with a pointed tip.

It is the goal of the invention to develop an improved application probe, in particular for such applications as the treatment of bronchial carcinomas.

According to the invention, this goal is achieved by an application probe which has a flexible, elongate hollow shank comprising a distal shank part, which shank extends along a longitudinal direction of the probe from a proximal shank end to a distal shank end. The distal shank part is formed by shank longitudinal sections, which lie successively in the longitudinal direction of the shank and of the probe, and are at least partly separated from one another in the longitudinal direction of the distal shank part. Shank longitudinal sections separated from one another in the longitudinal direction of the distal shank part means that the shank longitudinal sections are, for example, separated from one another along a separating line extending substantially in the circumferential direction of the distal shank part. Here, the separation, extending in the circumferential direction, of the shaft longitudinal sections from one another is such that they enable a flexible property of the distal shank part.

Moreover, the application probe has a pulling element, which has a proximal and a distal pulling element end, arranged in the interior of the shank. Finally, the application probe has a tensioning apparatus, which is connected to the proximal shank end and the proximal pulling element end, and enables selectively to tension the pulling element or leave it loose. To this end, the pulling element is connected to a longitudinal section of the distal shank part or to a probe tip connected to the distal shank end in such a way that the pulling element is to be tensioned by means of the tensioning apparatus and brings about such bracing of the shank longitudinal sections with one another in the tensioned state, that the bracing results in stiffening of the distal shank part as a result of reduced flexibility of the shank.

A preferably pliable proximal shank part, in which the pulling element can be guided, can adjoin the distal shank part on the proximal side thereof.

An application probe with such a shank setup can optionally assume a pliable or rigid state by means of the pulling element and the tensioning apparatus. The application probe is pliable when the pulling element is slack. The application probe becomes rigid when the pulling element is tensioned. For e.g. the bronchial carcinoma treatment, this is advantageous because the application probe is initially inserted into a work channel of an endoscope or the like in a slack state and can be advanced to the treatment location. When the application probe is then to penetrate e.g. a bronchial wall, it can initially be stiffened by tensioning the pulling element and it is then more suitable for penetrating a bronchial wall.

With respect to the structural design of the distal shank part, two embodiment variants, in particular, are of importance: firstly, the shank longitudinal sections can each be completely separated from one another, i.e. adjacent shank longitudinal sections are not integrally connected to one another. Secondly, the shank longitudinal sections can also be connected to one another in integral fashion, for example by material bridges, such that a separating line between the shank longitudinal sections does not extend around the complete circumference of the distal shank part. According to a preferred embodiment variant, the distal shank part is integral and the shank longitudinal sections are separated from one another by a separating line circumscribing the distal shank part along a helix, wherein the separating line is preferably selected in such a way that the separating line extending along the helix alternately proceeds back and forth in relation to the longitudinal direction of the shank. The wave-shaped profile prevents the individual shank longitudinal sections from being able to be displaced laterally with respect to one another.

Therefore, a profile of the separating line selected thus is also advantageous if the separating line, in principle, extends around the distal shank part in the circumferential direction and completely separates the individual shank longitudinal sections from one another.

A further advantageous embodiment of the distal shank part with shank longitudinal sections completely separated from one another is configured in such a way that the shank longitudinal sections engage in one another in the radial direction, for example by virtue of a tapering end face of a shank longitudinal section sliding into a corresponding end face of an adjacent shank longitudinal section. While the shank longitudinal sections therefore engage in one another in the circumferential direction in the embodiment variant with a separating line extending in a wave-shaped manner, the shank longitudinal sections engage in one another in the radial direction in the variant mentioned last, and are thus secured against lateral offset with respect to one another.

According to a preferred embodiment variant, one or more shank longitudinal sections consist of electrically insulating material and act as insulation element.

The latter is advantageous, in particular, if one or more shank longitudinal sections are electrically conductive, at least on the outer side thereof, and are embodied as electrode for applying electric current to a medium surrounding the probe during use. Then, the distal shank part can act like a unipolar or bipolar ablation probe, known per se, the electrodes of which are formed by the electrically conductive shank longitudinal sections and which are electrically insulated from one another by the electrically nonconductive shank longitudinal sections, which act as insulators.

An optionally provided probe tip can form at least one part of a distal ablation electrode of the application probe and the pulling element can be embodied as electric conductor, by means of which the distal electrode, formed by electrically conductive shank longitudinal sections, is to be connected electrically to a current or voltage source.

The probe tip is preferably a pointed tip with e.g. trocar grinding or conical, or similar, grinding, in order e.g. to be able to penetrate a cartilaginous bronchial wall.

To this end, the probe tip can also have a cutting electrode, which can be employed for electrosurgical cutting by applying a radiofrequency AC voltage. During electrosurgical cutting, a spark discharge emanates from the cutting electrode, which spark discharge burns or vaporizes the tissue around the cutting electrode and thereby enables cutting of tissue with little hemorrhaging. Instead of having a special cutting electrode, the probe tip can also be embodied as a cutting electrode, for example by virtue of being pointed toward the distal end thereof.

In particular, the pulling element as pulling wire can, in this case, be made of an electrically conductive material such that the pulling element can electrically connect the cutting electrode to a current or voltage source.

In a preferred embodiment, the pulling wire is surrounded by an electrically insulating insulation sleeve, which can e.g. be a pliable tube pulled over the pulling wire. The insulation sleeve can consist of an insulating material, e.g. a plastic, for example polyvinylidenefluoride (PVDF), polyetheretherketone (PEEK) or the like. There can be a clearance between pulling wire and insulation sleeve, which clearance guides a fluid for temperature regulation, e.g. for cooling the electrodes. In this case, an outer insulation sleeve forms a return channel for the fluid, while the fluid is guided to the electrode through a channel lying in the inner insulation sleeve.

The proximal shank part can have an electrically insulating outer insulation sleeve and contain a (second) electric conductor which electrically contacts a proximal ablation electrode formed by electrically conductive shank longitudinal sections. By way of example, the second electric conductor can be configured as a wire helix, which supports the outer insulation sleeve from the inside but nevertheless is pliable. Such a wire helix, like a Bowden cable, has the advantage that it can take up pressure forces acting in the axial direction of the proximal shank part.

In a further advantageous embodiment, the probe tip can be guided in a shank longitudinal section, wherein the tension of the pulling element simultaneously leads to the probe tip extending out of the shank longitudinal section. As a result, a pointed probe tip can be guided in the pliable state to a treatment location, without damaging an endoscope or injuring tissue, wherein the application probe then gains its functionality at said treatment location by tensioning the pulling element.

Additionally, or alternatively, the distal shank part can contain a phase change material (PCM), for example a salt-containing solution, such as sodium acetate trihydrate, or other phase change materials which change between liquid and solid phase.

The phase change materials preferably change between a first pliable state and a second rigid state by the activation and supply of energy via the shank and/or the electrically conductive pulling wire.

The invention is now intended to be explained in more detail using exemplary embodiments schematically depicted in the figures. In the figures:

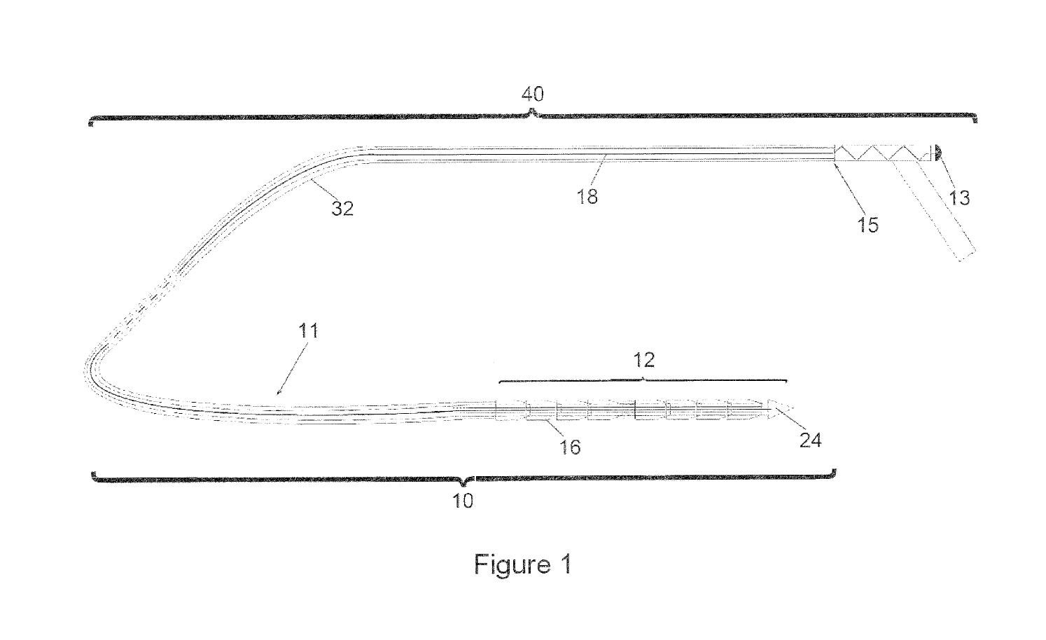

FIG. 1 shows a schematic illustration of an application probe.

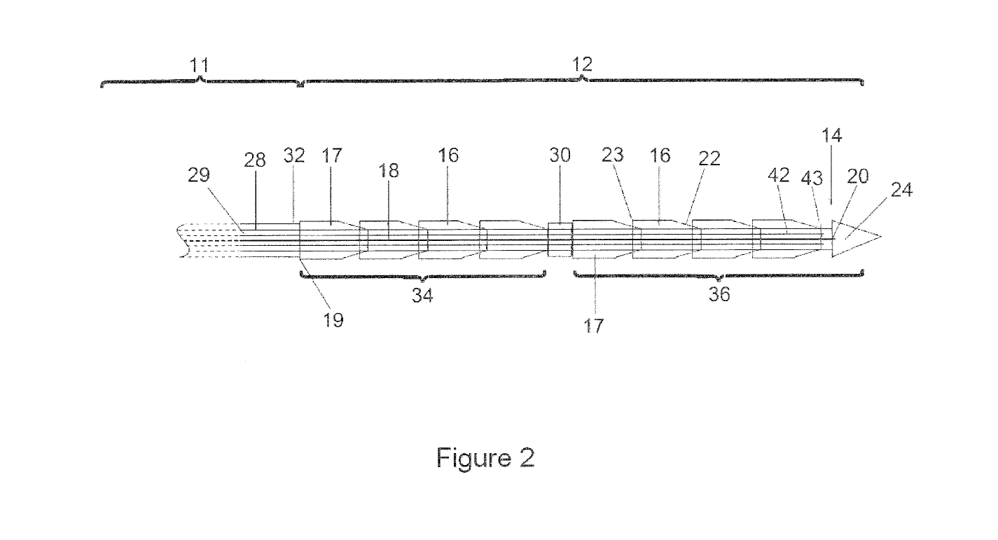

FIG. 2 shows, in a longitudinal section, a schematic illustration through a first exemplary embodiment of the distal shank part of an application probe in the pliable state.

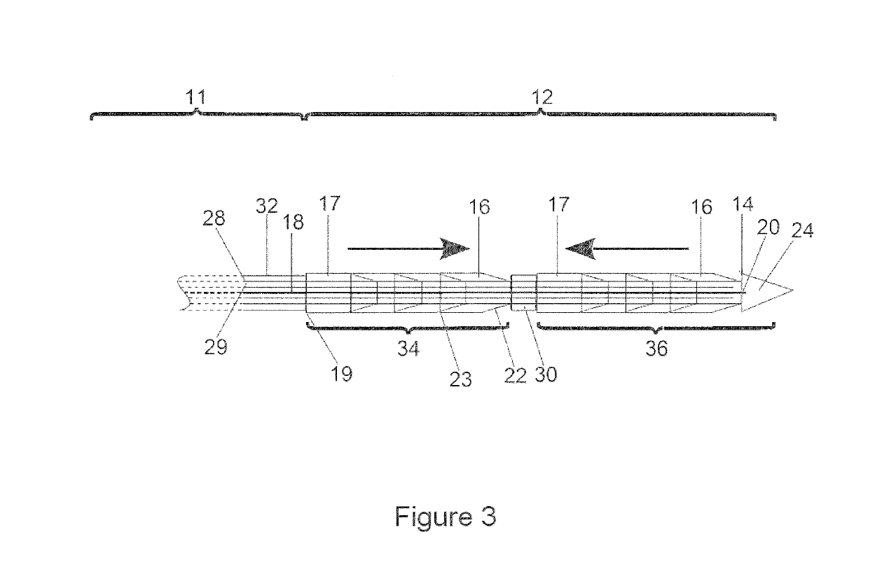

FIG. 3 shows, in a longitudinal section, a schematic illustration through a first exemplary embodiment of the distal shank part of an application probe in the rigid state.

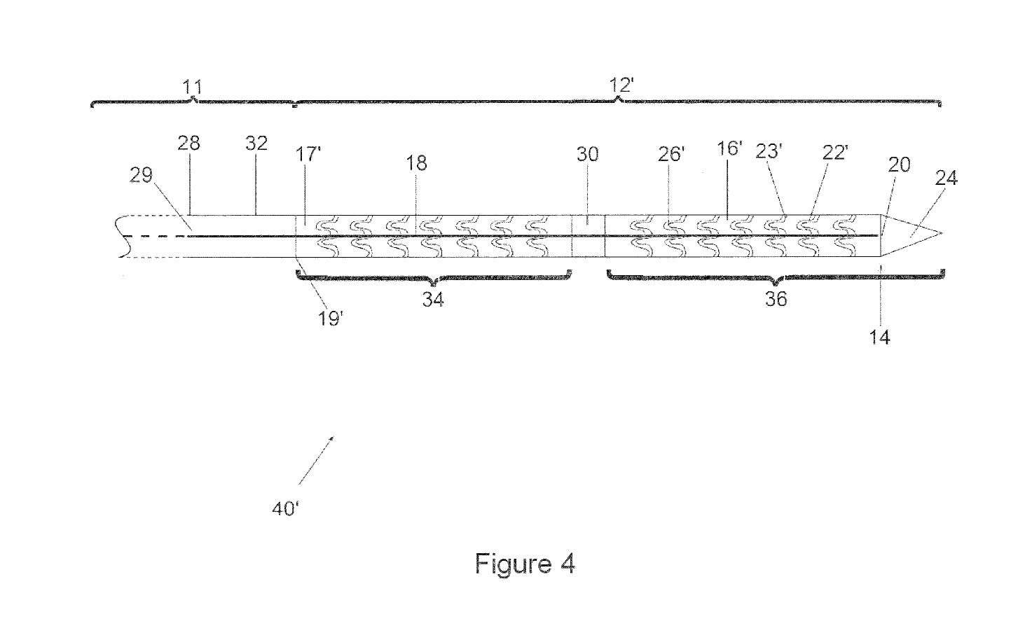

FIG. 4 shows a schematic illustration of a second exemplary embodiment of the distal shank part of an application probe in the pliable state.

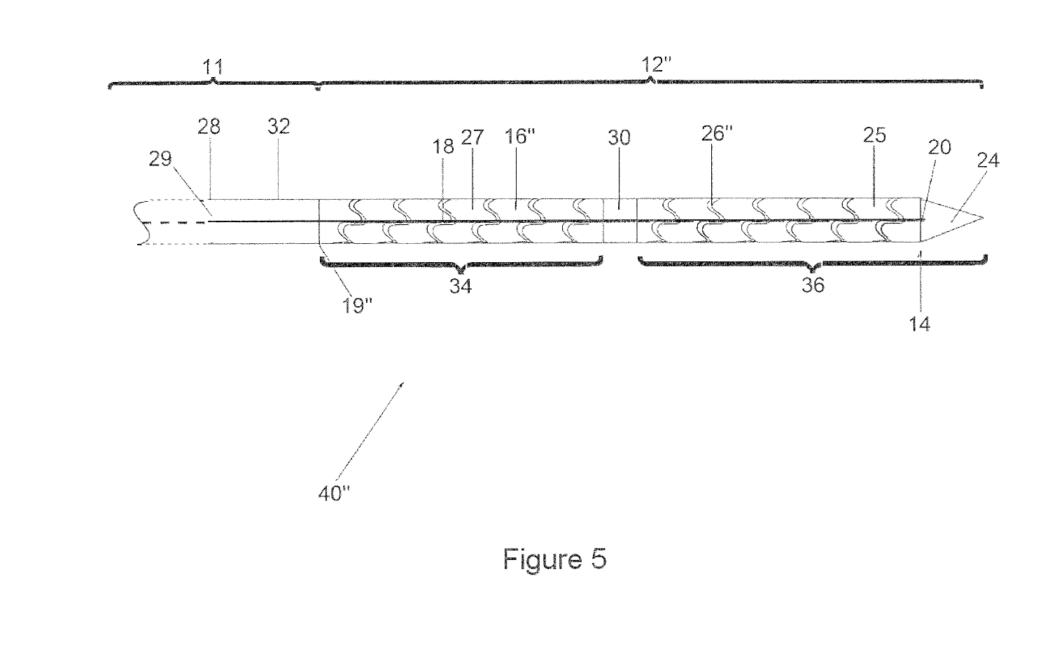

FIG. 5 shows a schematic illustration of a third exemplary embodiment of the distal shank part of an application probe in the pliable state.

FIG. 1 shows a schematic illustration of a first exemplary embodiment of an application probe 40 according to the invention. The application probe 40 has a flexible, elongate, hollow shank 10 extending along a longitudinal direction of the probe, which shank comprises two shank parts connected to one another, namely a proximal shank part 11 and a distal shank part 12. At the proximal shank end 15 thereof, the shank 10 is connected to a tensioning apparatus 13, and said shank has a probe tip 24 at its distal shank end.

The proximal shank part 11 is pliable and has an electrically insulating outer insulation sleeve 32.

In the interior of the shank 10, a pulling element 18 extends from the tensioning apparatus 13 to one of the shank longitudinal section 16 or the probe tip 24. By means of the tensioning apparatus 13, it is possible, alternatively, to tension the pulling element 18, e.g. a pulling wire or the like, or leave it loose.

The distal shank part 12 has shank longitudinal sections 16 connected to one another in a flexible manner and can assume two states, namely a first, non-tensioned, comparatively flexible state and a second, tensioned and comparatively rigid state.

Pliability describes one state of the shank 10, in which the latter has a flexibility enabling the shank to be guided along curved lumens, which e.g. only have an insubstantially larger internal diameter than the external diameter of the shank 10. The distal shank part 12 is pliable in the first state thereof (FIG. 2), enabling the shank to be guided to the treatment location via curved lumens, e.g. hollow organs or endoscopes guided in hollow organs or other curved lumens.

For establishing the second, comparatively rigid state of the distal shank part, the pulling element 18 is put under tension by the tensioning apparatus 13, e.g. a spring, a tensioning screw or the like, as a result of which the shank longitudinal sections 16 of the distal shank part 12 brace against one another such that the distal shank part 12, as a result thereof, is rigid and can be employed to e.g. penetrate a tissue wall such as e.g. a cartilaginous bronchial wall or the like.

FIG. 2 shows a schematic illustration, as a longitudinal section, through the first exemplary embodiment of the distal shank part 12 of the application probe 40 according to the invention in the first (pliable) state. Along the longitudinal direction of the shank 10, the distal shank part 12 has successive shank longitudinal sections 16 and at least one shank longitudinal end section 17, which are at least partly separated from one another in the circumferential direction of the shank 10. The separation, extending in the circumferential direction, of the shank longitudinal sections 16, 17 from one another is such that they enable a flexibility of the shank 10.

In the first exemplary embodiment, the shank longitudinal sections 16 completely separated from one another in the circumferential direction are embodied with two different end faces 22, 23 to enable flexibility. The distal end faces 22 (in the exemplary embodiment) of the shank longitudinal sections 16 taper along the longitudinal axis, while, in the exemplary embodiment, the proximal end faces 23 are embodied to receive a tapering end face 22 of an adjacent shank longitudinal section 16. As a result, the tapering shank ends 22 can be inserted into the receiving end faces 23 of the shank longitudinal sections 16, which have a complementary fit to the tapering distal end faces of the shank longitudinal sections 22. In embodiment variants (not depicted here), it is for example also possible for shank longitudinal sections with two receiving end faces to alternate with shank longitudinal sections with two tapering end faces. It is also possible for the tapering end faces to point in the proximal direction and the receiving shaft ends to point in the distal direction.

In the pliable state of the distal shank part 12, the shank longitudinal sections 16 or the tapering distal end faces 22 are inserted, not under tension, into the respective adjoining shank longitudinal sections 16 thereof, i.e. the shank longitudinal sections 16 project into the respectively adjacent shank longitudinal sections 16 but can move relative to one another both along the longitudinal axis and also, to a certain extent, perpendicular to the longitudinal axis, as result of which the distal shank part 12 is pliable.

The tapering distal end faces 22 of the shank longitudinal sections 16 can have end areas with different shapes, e.g. convex-spherically shaped end areas, concavely spherically with, complementary thereto, convex-spherically shaped adjacent end areas, planar end areas extending transversely to the longitudinal direction of the shank 10, conical frustum-shaped end areas, conically shaped end areas or the like. It is to be noted here that the adjacent receiving end face 23 of the shank longitudinal section 16 has a complementary fit to the respective tapering distal end face 22 of the shank longitudinal section 16 so that the shank longitudinal sections 16 can be pushed into one another or so that the proximal end face 23 can receive the distal end face 22.

The shank longitudinal end section(s) 17 has/have a proximal end face 19, which is connected to the proximally adjacent material thereof in such a way that a secure connection, i.e. a connection which is not flexible at the proximal end of the shank longitudinal end section 17, is created, wherein the shank longitudinal end section 17 therefore only has flexibility at the distal end face 22. The shank longitudinal end section 17 can also be embodied with a receiving proximal end face 23, wherein in a second (rigid) state, the connection between shank longitudinal end section 17 and proximally adjacent material is fixed. In the exemplary embodiment, the shank longitudinal end section 17 is securely connected, via the proximal end 19 thereof, to the pliable outer insulation sleeve 32 of the proximal shank part 11 and therefore forms a connection between the proximal shank part 11 and the distal shank part 12.

The shank longitudinal sections 16 coaxially surround an inner pliable insulation sleeve 28, which contains an inner channel 29 with the pulling element 18, wherein, in the first exemplary embodiment, the pulling element 18 has a pulling wire 18. The pliable insulation sleeve 28 can have a probe tip 24, with which the pulling wire 18 has a tension-resistant connection via the pulling element end 20 thereof. Alternatively, the pulling wire 18 can also have a tension-resistant connection (not depicted here) with a shank longitudinal section 16. In this exemplary embodiment, the probe tip 24 is configured as a pointed tip, e.g. with trocar grinding or conically shaped, or similar, grinding.

The pulling wire 18 can consist of an electrically conductive material, e.g. an alloy such as e.g. AlMg5 or the like, and can connect the distal shank end 14, e.g. the probe tip 24 or the shank longitudinal sections 16, to a current or voltage source. To this end, the probe tip 24 can be embodied as cutting electrode or can have a cutting electrode, to which a radiofrequency AC voltage can be applied for electrosurgical cutting (not shown here). In order to achieve shielding of the current flows, the pliable insulation sleeve 28 is made of an electrically insulating, reversibly deformable material, e.g. a plastic such as e.g. polyvinylidenefluoride (PVDF), polyetheretherketone (PEEK) or the like, which ideally does not increase the rigidity of the application probe where possible.

In the shown exemplary embodiment, the shank longitudinal sections 16 are configured as bipolar electrodes 34, 36 for applying electric current to a medium surrounding the probe during use, i.e. the shank longitudinal sections 16, however at least parts of the surface thereof, consist of an electrically conductive material, e.g. an alloy, such as e.g. steel or the like, or a metal, such as e.g. iron, gold, silver or the like, wherein care has to be taken that the material is biocompatible with human tissue and hard enough to penetrate human tissue. The proximal electrode 34 is separated from the distal electrode 36 by an insulation element 30, wherein the insulation element 30 is not electrically conductive and therefore insulates the bipolar electrodes 34, 36 from one another. The insulation element 30 can be configured with a compatible fit to the shank longitudinal sections 16, as a result of which the insulation element 30 is identical to the shank longitudinal sections 16 apart from the insulating material from which it consists (not shown here). In the shown exemplary embodiment (FIG. 2), the insulation element 30 has a non-compatible fit with the shank longitudinal sections 16, wherein the shank longitudinal section 16 distally adjacent to the insulation element 30 in this case is an shank longitudinal end section 17 with the proximal end face 19, which is fixedly, within the meaning of what has been said, connected to the insulation element 30. The distal shank part can also have a plurality of insulation elements 30 which produce multipolar electrodes (not shown here). In the shown exemplary embodiment, the probe tip 24 forms one part of the distal electrode 36.

An inner channel 29, which is enclosed by the pliable insulation sleeve 28, can contain a fluid or the like, which where possible does not increase the rigidity of the application probe and e.g. can be employed for temperature regulation of the application probe, in particular for cooling the electrodes 34, 36. To this end, the fluid is routed distally out of the fluid channel 42 into the inner channel 29 through the inflow 43, in order thus to enable circulation of the fluid. The fluid then flows along the inner channel 29 proximally to the end of the probe. The pressure in the distal shank part 12 can be increased (not shown here) by a restrictor, e.g. butterfly valve, reducer or the like, which is arranged in the inner channel 29. The increased pressure in the inner channel 29 then expands the insulation sleeve 28, as a result of which the shank longitudinal sections 16, 17 are restricted in terms of the radial flexibility thereof and the distal shank part 12 obtains additional rigidity.

The electrically insulating pliable outer insulation sleeve 32 coaxially surrounds the inner electrically insulating pliable insulation sleeve 28 in the proximal shank part 11 and consists of electrically insulating material, which is compatible with the material of the inner pliable insulation sleeve 28 and of a possibly surrounding endoscope, or with human or animal tissue. The shank longitudinal end section 17 can be connected to a conducting medium, e.g. wire, conductive layer or a similar current conducting means, which is situated outside of the inner pliable insulation sleeve 28 and within the pliable outer insulation sleeve 32, and serves to complete the current circuit. Alternatively, the distal electrode 36 can also have a monopolar configuration, wherein the current circuit is closed by a large-area return or neutral electrode lying against the body of the patient.

The diameter of the shank 10, and hence also the application probe 40, is less than 3 mm (9 French), for example 2 mm (6 French), for example 1.8 mm (5-6 French), and so this can optionally be guided through an endoscope, e.g. bronchoscope, gastroscope or the like, or directly through human or animal tissue or lumens into human or animal bodies.

FIG. 3 shows a schematic illustration, as a longitudinal section, through the first exemplary embodiment of the distal shank part 12 of the application probe 40 according to the invention in a second (rigid) state. The pulling wire 18 is under mechanical tension and, via the probe tip 24, exerts pressure force, acting axially, on the shank longitudinal sections 16, the fixed shank longitudinal end section 17, which are thereby braced against one another. The shank longitudinal end section 17 is pressed against a distal end face of the outer insulation sleeve 32 by the pressure force, which outer insulation sleeve can receive pressure forces acting in the axial direction, produces a counterforce, which enables the bracing of the shank longitudinal sections against one another.

In the tensioned state of the pulling element, the shank longitudinal sections 16, the probe tip 24 and the shank longitudinal end section 17 are completely, or almost completely, inserted into the respective adjacent shank longitudinal sections 16 thereof and therefore cannot be displaced, neither along the longitudinal axis, nor perpendicular to the longitudinal axis, as a result of which the distal shank part 12 is rigid. In the rigid state (FIG. 3), the length of the distal shank part 12 is reduced compared to the length of the distal shank part 12 in the pliable state (FIG. 2), wherein the insulation sleeve 28 lying coaxially around the pulling wire 18 is displaced relative to the outer insulation sleeve 32 and is pulled out of the shank 10 together with the pulling wire 18 by the tensioning apparatus 13 (not shown here).

FIG. 4 shows a schematic illustration of the first (pliable) state of a second exemplary embodiment of a distal shank part 12 of an application probe 40′ according to the invention. The application probe 40′ as per FIG. 4 differs from the application probe as per FIGS. 1, 2 and 3 only in terms of the distal shank part 12′ thereof. The distal shank part 12′ of the second exemplary embodiment is similar to the distal shank part 12 of the first exemplary embodiment (FIG. 2), wherein, in particular, the shape and the connections of the shank longitudinal sections 16′ differ from the exemplary embodiment in FIGS. 1 to 3. The shank longitudinal sections 16′ have a distal end face 22′ and a proximal end face 23′ with wavy separating lines 26′, which are configured in such a way that respectively adjacent shank longitudinal sections 16′ engage into one another in such a way that, when the pulling element 18 is tensioned, a rigid or non-flexible connection is produced between adjacent shank longitudinal sections 16′, as a result of which the distal shank part 12′ is non-flexible or rigid. In a first state, the pulling element 18 is not under tension, as a result of which the shank longitudinal sections 16′ are arranged in such a way that they will lie on one another in such a way that they can be tilted but not laterally displaced relative to one another, as a result of which the distal shank part 12′ is pliable in the first state of the second exemplary embodiment. In a second state, the pulling element 18 is tensioned, as a result of which the shank longitudinal sections 16′, like in the first exemplary embodiment, lie on one another on their end areas under pressure (and not only loosely) from the probe tip 24 and the shank longitudinal end section 17′, and the distal shank part 12′ is rigid (not shown here).

FIG. 5 shows a schematic illustration of the first (pliable) state of a third exemplary embodiment of a distal shank part 12″ of an application probe 40″ according to the invention. The distal shank part 12″ differs from the preceding exemplary embodiments (FIG. 1 and FIG. 3) in terms of the shank longitudinal sections 16″, which each form sections (i.e. one of a plurality of windings) of a helix. In the depicted exemplary embodiment, two helices are shown, of which respectively one helix extends over the whole length of one of the electrodes 34, 36. In the third exemplary embodiment, a distally circumscribing helix 25 together with a probe tip 24 forms a distal electrode 36. The separating line 26″, extending in the circumferential direction of the distal shank part 12″ in a helical manner, is wavy, and so it alternately proceeds back and forth in the longitudinal direction of the distal shank part 12″. The proximal electrode 34 is likewise formed by proximal helix 27, the windings of which each form a shank longitudinal section 16″. The helices 25 and 27, and therefore the proximal electrode 34 and the distal electrode 36, are electrically separated from one another by an insulation element 30.

### LIST OF REFERENCE SIGNS

10 Shank
1 Proximal shank part
12 Distal shank part
13 Tensioning apparatus
14 Distal shank end
15 Proximal shank end
16 Shank longitudinal section
17 Shank longitudinal end section
18 Pulling wire
19 Proximal end face of a shank longitudinal end section
20 Distal pulling element end
22 Distal end face
23 Proximal end face
24 Probe tip
25 Distal helix
26 Wavy separating line
27 Proximal helix
28 Pliable insulation sleeve
29 Inner channel
30 Insulation element
32 Pliable outer insulation sleeve
34 Proximal electrode
36 Distal electrode
40 Application probe
42 Fluid channel
43 Inflow

1. Application probe, comprising
a flexible, elongate, hollow shank extending along a longitudinal direction of the probe from a proximal shank end to a distal shank end, said shank comprising a distal shank part which is formed by shank longitudinal sections which lie successively in the longitudinal direction of the distal shank part and of the probe, said shank longitudinal sections being at least partly separated from one another in the circumferential direction of the distal shank part, wherein the separation, extending in the circumferential direction, of the shank longitudinal sections from one another is such that this enables a flexibility of the distal shank part, said application probe further comprising
a pulling element arranged in the interior of the distal shank part, comprising a proximal pulling element end and a distal pulling element end, said application probe further comprising
a tensioning apparatus connected to the proximal shank end and the proximal pulling element end,
wherein said distal pulling element end is connected to either one of said shank longitudinal sections of the distal shank part or to a probe tip connected to the distal shank end in such a way that the pulling element is to be tensioned by means of the tensioning apparatus and brings about bracing of the shank longitudinal sections with one another in the tensioned state, which bracing results in stiffening of the distal shank part as a result of reduced flexibility.

2. Application probe according to claim 1, wherein the shank longitudinal sections are separated from one another along a separating line circumscribing the distal shank part along a helix.

3. Application probe according to claim 2, wherein the separating line is wavy along the helix and therefore alternately proceeds back and forth in the longitudinal direction of the distal shank part.

4. Application probe according to claim 1, wherein the shank longitudinal sections are in each case completely separated from one another in the circumferential direction of the distal shank part.

5. Application probe according to claim 4, wherein at least one or some shank longitudinal sections have at least one convex-spherically shaped end area, which extends into a respectively adjacent shank longitudinal section.

6. Application probe according to claim 5, wherein adjacent shank longitudinal sections respectively have a concave-spherically shaped end area and a convex-spherically shaped end area which is complementary thereto.

7. Application probe according to claim 4, wherein adjacent shank longitudinal sections each have planar end areas extending transversely to the longitudinal direction of the shank.

8. Application probe according to claim 4, wherein at least one or some shank longitudinal sections have at least one conical frustum-shaped end area, which respectively extends into a respectively adjacent shank longitudinal section.

9. Application probe according to claim 1, wherein the pulling element has a pulling wire.

10. Application probe according to claim 9, wherein the application probe has a probe tip at the distal end thereof, which probe tip has a tension-resistant connection to the pulling wire.

11. Application probe according to claim 1, wherein one or more shank longitudinal sections consist of electrically insulating material and act as insulating element.

12. Application probe according to claim 1, wherein one or more shank longitudinal sections are electrically conductive, at least on the outer side thereof, and are embodied as electrode for applying electric current to a medium surrounding the probe during use.

13. Application probe according to claim 10, wherein the probe tip forms at least one part of a distal electrode of the application probe and in that the pulling wire is embodied as electric conductor, by means of which the distal electrode is to be connected electrically to a current or voltage source.

14. Application probe according to claim 10, wherein the probe tip has a pointed tip with e.g. trocar grinding or conical, or similar, grinding.

15. Application probe according to claim 13, wherein the probe tip has a cutting electrode or is embodied as cutting electrode, to which a radiofrequency AC voltage can be applied for electrosurgical cutting.

16. Application probe according to claim 1, wherein the diameter of the shank, and hence also the application probe, is less than 3 mm (9 French).
